(19) United States
(12) Patent Application Publication
(10) Pub. No.: US 2024/0017961 A1
(43) Pub. Date: Jan. 18, 2024

(54) AUTOMATED OPTICAL SYSTEM FOR DETECTION OF A BUTTON SANITARY CONDITION AND CORRESPONDING METHOD

(71) Applicant: TECHNOLOGIES GRB INC., Quebec (CA)

(72) Inventors: Raymond Boisvert, Quebec (CA); Pierre Marquet, Stoneham-et-Tewkesbury (CA); Andre Villemaire, Quebec (CA)

(21) Appl. No.: 18/032,049

(22) PCT Filed: Oct. 12, 2021
(86) PCT No.: PCT/CA2021/051429

§ 371 Date: Apr. 14, 2023

Related U.S. Application Data
(60) Provisional application No. 63/092,022, filed on Oct. 15, 2020.

(51) Int. Cl. B66B 1/46; A61L 2/10; G01N 21/94; G01N 21/45; G01N 21/47

(52) U.S. Cl. CPC B66B 1/466; A61L 2/10; G01N 21/94; G01N 21/453; G01N 21/4788; A61L 2202/14

(57) ABSTRACT

The present disclosure concerns an automated optical system for detection of a sanitary condition of a button, the system comprising a button housing defining a button-receiving cavity and comprising an inner side; a button disposed in the button-receiving cavity; an optical assembly to capture an optical signal of the button; a sanitary condition detection assembly to detect a predetermined sanitary condition of the button from information derived from the captured optical signal; and a disinfecting system for disinfecting the button. It also concerns a corresponding method.

### PRIOR APPLICATION

The present application claims priority from U.S. provisional patent application No. 63/092,022, filed on Oct. 15, 2020, and entitled “AUTOMATED OPTICAL SYSTEM FOR DETECTION OF A BUTTON SANITARY CONDITION AND CORRESPONDING METHOD”, the disclosure of which being hereby incorporated by reference in its entirety.

### TECHNICAL FIELD

The technical field relates to systems for detection of a sanitary condition, and more particularly to automated optical systems for detection of a sanitary condition of a button and corresponding methods.

### BACKGROUND

Elevators are convenient apparatus for humans working or living in high-rise buildings. Modern elevators carry people and goods on any floor rapidly and securely. Most elevators use control systems that respond primarily to floor requests by way of buttons installed on user panels. The vast majority of these panels actually requires the user to physically touch and press the buttons, allowing germs and bacteria to be transferred from hands of the users to the buttons and from the buttons to the hands. It is indeed known that direct hand contact is one important method by which germs and bacteria spread through the population and that contributes to increased risk of contamination and disease.

Buttons, such as elevator buttons, may thus constitute pathogen dissemination sources, which may result in a spreading of infectious diseases, in particular when the elevator equips hospitals and/or health centers. It is in particular described in Kandet et al. “Elevator buttons as unrecognized sources of bacterial colonization in hospitals”, 2014. Usually, a detection of pathogens on a button is carried out manually (for instance via samples which are sent to a laboratory for analysis).

In view of the above, there is a need for a button sanitary condition detection system which would be able to overcome or at least minimize some of the above-discussed prior art concerns, and to a corresponding method.

### BRIEF SUMMARY

It is therefore an aim of the present invention to address the above-mentioned issues.

According to a general aspect, there is provided an automated optical method for detecting a sanitary condition on a button, the method comprising capturing at least one optical signal of at least one of physical and electrical features of the button; detecting a particular sanitary condition of the button from information derived from said at least one captured optical signal of said at least one of physical and electrical features of the button; and disinfecting at least partially the button.

According to another general aspect, there is provided an automated optical method for detecting a sanitary condition on a button, the method comprising: capturing at least one optical signal of at least one of physical and electrical features of the button; determining whether the button has a particular sanitary condition based on information derived from said at least one captured optical signal of said at least one of physical and electrical features of the button; and disinfecting at least partially the button.

According to another general aspect, there is provided an automated optical detection system for detection of a sanitary condition of a button, the system comprising: a button housing defining a button-receiving cavity and comprising an inner side; at least one button disposed at least partially in the button-receiving cavity; an optical assembly to capture at least one optical signal of at least one of physical and electrical features of the button; a sanitary condition detection assembly to detect a predetermined sanitary condition of the button from information derived from said at least one captured optical signal of said at least one of physical and electrical features of the button; and a disinfecting system for disinfecting the button.

### BRIEF DESCRIPTION OF THE DRAWINGS

FIG. 1 is a top perspective view illustrating a user pressing a button in an elevator;

FIG. 2 is a block diagram representing the different steps of a method for detecting a sanitary condition on a button;

FIG. 3 is a front elevational view of an automated optical detection system in accordance with an embodiment, the system comprising a button housing defining a button-receiving cavity, the system further comprising a button in the button-receiving cavity and being at least partially reachable from an outside of the button housing;

FIG. 4 is a bottom perspective view of the system of FIG. 3, the button being removed;

FIG. 5 is a sectional view taken along cross-section lines 5-5 of the automated optical detection system of FIG. 3;

FIG. 6 is a bottom perspective view of the optical detection system of FIG. 3, a front panel of the button housing being removed; and

FIG. 7 is a bottom perspective view of the optical detection system of FIG. 6, a printed circuit board and the button being removed.

### DETAILED DESCRIPTION

In the following description, the same numerical references refer to similar elements. Furthermore, for the sake of simplicity and clarity, namely so as to not unduly burden the figures with several references numbers, not all figures contain references to all the components and features, and references to some components and features may be found in only one figure, and components and features of the present disclosure which are illustrated in other figures can be easily inferred therefrom. The embodiments, geometrical configurations, materials mentioned and/or dimensions shown in the figures are optional and are given for exemplification purposes only. Moreover, it will be appreciated that positional descriptions such as “above”, “below”, “forward”, “rearward”, “left”, “right” and the like should, unless otherwise indicated, be taken in the context of the figures only and should not be considered limiting. Moreover, the figures are meant to be illustrative of certain characteristics of the automated optical detection system and are not necessarily to scale. To provide a more concise description, some of the quantitative expressions given herein may be qualified with the term “about”. It is understood that whether the term “about” is used explicitly or not, every quantity given herein is meant to refer to an actual given value, and it is also meant to refer to the approximation to such given value that would reasonably be inferred based on the ordinary skill in the art, including approximations due to the experimental and/or measurement conditions for such given value. In the following description, an embodiment is an example or implementation. The various appearances of “one embodiment”, “an embodiment” or “some embodiments” do not necessarily all refer to the same embodiments. Although various features may be described in the context of a single embodiment, the features may also be provided separately or in any suitable combination. Conversely, although the invention may be described herein in the context of separate embodiments for clarity, it may also be implemented in a single embodiment. Reference in the specification to “some embodiments”, “an embodiment”, “one embodiment” or “other embodiments” means that a particular feature, structure, or characteristic described in connection with the embodiments is included in at least some embodiments, but not necessarily all embodiments.

It is to be understood that the phraseology and terminology employed herein is not to be construed as limiting and are for descriptive purpose only. The principles and uses of the teachings of the present disclosure may be better understood with reference to the accompanying description, figures and examples. It is to be understood that the details set forth herein do not construe a limitation to an application of the disclosure. Furthermore, it is to be understood that the disclosure can be carried out or practiced in various ways and that the disclosure can be implemented in embodiments other than the ones outlined in the description above. It is to be understood that the terms “including”, “comprising”, and grammatical variants thereof do not preclude the addition of one or more components, features, steps, or integers or groups thereof and that the terms are to be construed as specifying components, features, steps or integers. If the specification or claims refer to “an additional” element, that does not preclude there being more than one of the additional element. It is to be understood that where the claims or specification refer to “a” or “an” element, such reference should not be understood as meaning that there is only one of that element. It is to be understood that where the specification states that a component, feature, structure, or characteristic “may”, “might”, “can” or “could” be included, that particular component, feature, structure, or characteristic is not required to be included. The descriptions, examples, methods and materials presented in the claims and the specification are not to be construed as limiting but rather as illustrative only. Meanings of technical and scientific terms used herein are to be commonly understood as by one of ordinary skill in the art to which the invention belongs, unless otherwise defined. It will be appreciated that the methods described herein may be performed in the described order, or in any suitable order.

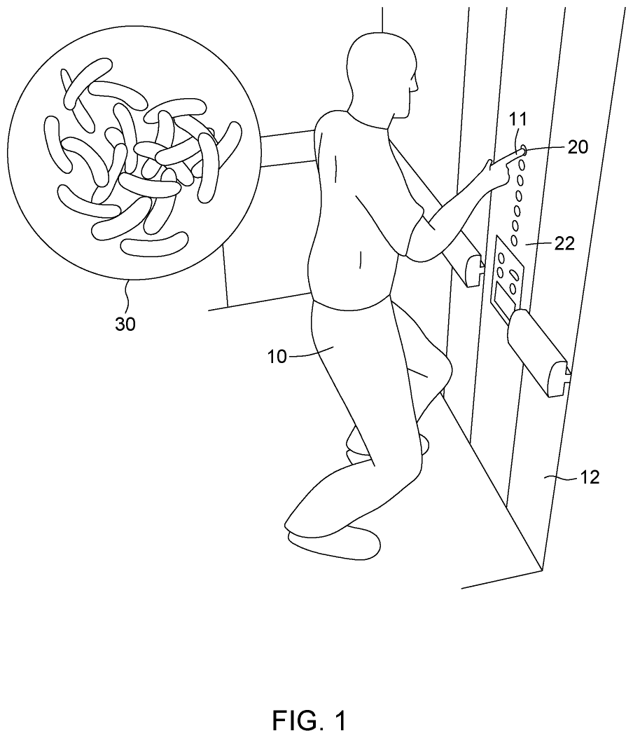

FIG. 1 shows a person 10 inside an elevator 12. The person 10 uses one of his fingers 11 to press on a button 20 located on an elevator button panel 22. The physical contact of the finger 11 and the button 20 allows germs and bacteria 30 to be exchanged from the finger 11 to the button 20 and from the button 20 to the finger 11. For instance, the elevator 12 comprises a plurality of buttons which can be assigned to building floors or other elevator functions such as open/close door and alarm call. In the embodiment shown, the buttons are used to inform the elevator system of a request to go to a particular floor. Corresponding floor indicators can also be provided that can be made of translucent material and be illuminated from a rear of the panel 22 using low intensity lights such as LED in order to inform the user that the floor call has been registered.

In other words, bacteria and/or viruses which may be found on the buttons have previously contaminated users and thus pathogens which are detected onto the buttons form representative samples of pathogens that can be found on the users. In yet other words, the pathogens that can be found on the buttons are representative samples of the pathogens that can be found in the building equipped with the elevator. Users could also be explicitly required to press thereon, in order to track circulation of pathogens within the equipped building. As detailed below, the present invention discloses an automated optical detection system for detection of a sanitary condition of an elevator button and an automated optical method for detecting a sanitary condition on an elevator. The same systems and methods could also be applied to other user panels where humans press elements including but not limited to control buttons, machine actuation buttons, ATM buttons, flushing buttons, doorbells, pedestrian crossing buttons, museum interactive display panels, control panels in industrial settings, public phones, internet cafés, keyboards and the like, in hospitals, cruise ships, transport industry, public places and the like. It could also be used to control human circulations in cities or public places, as a function of detected pathogen circulations within the corresponding cities or public places.

### Automated Optical Detection System

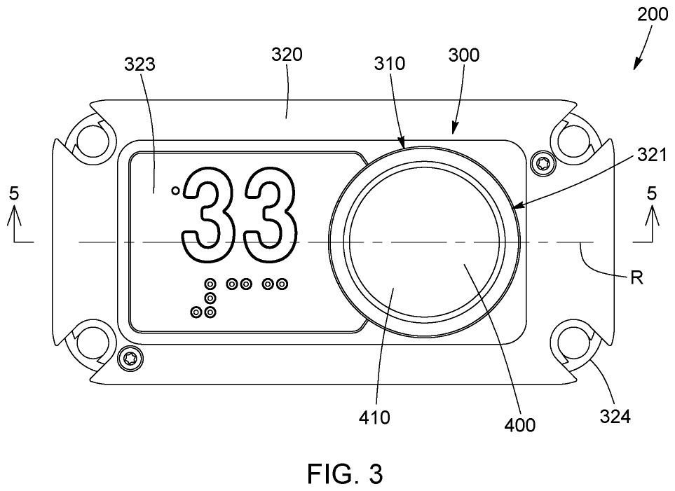

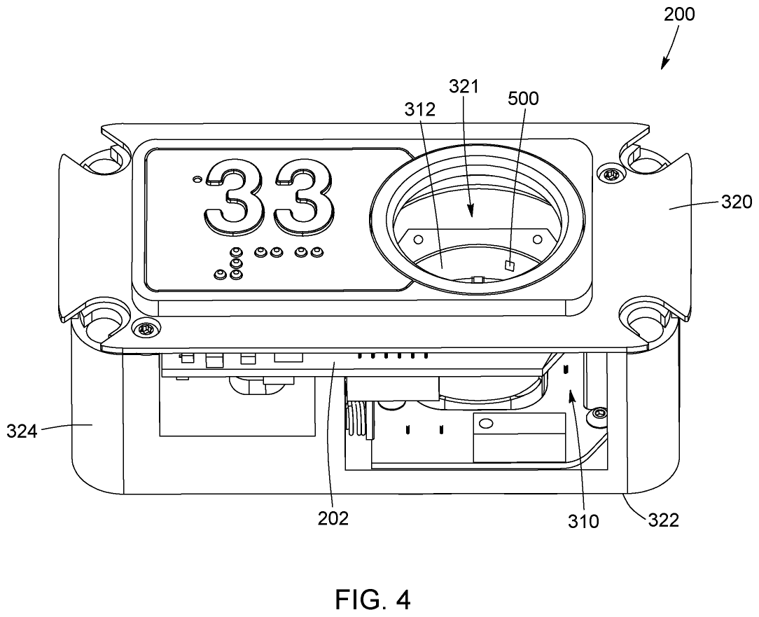

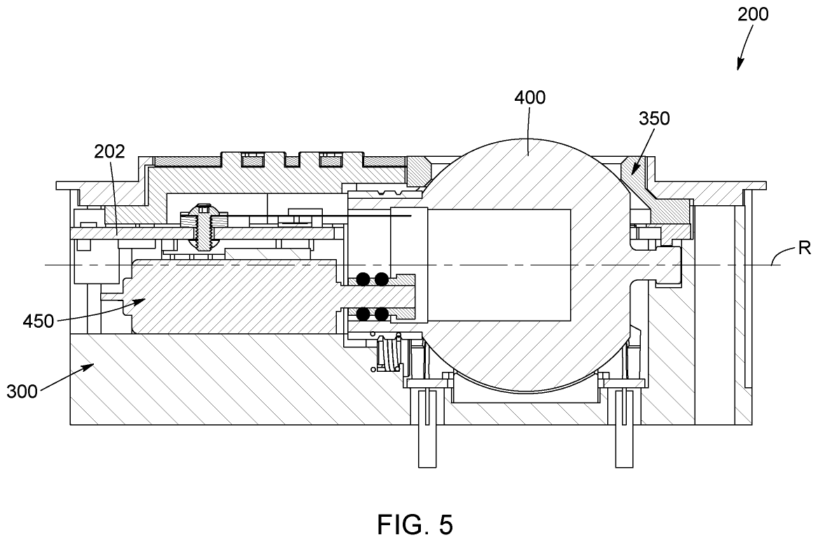

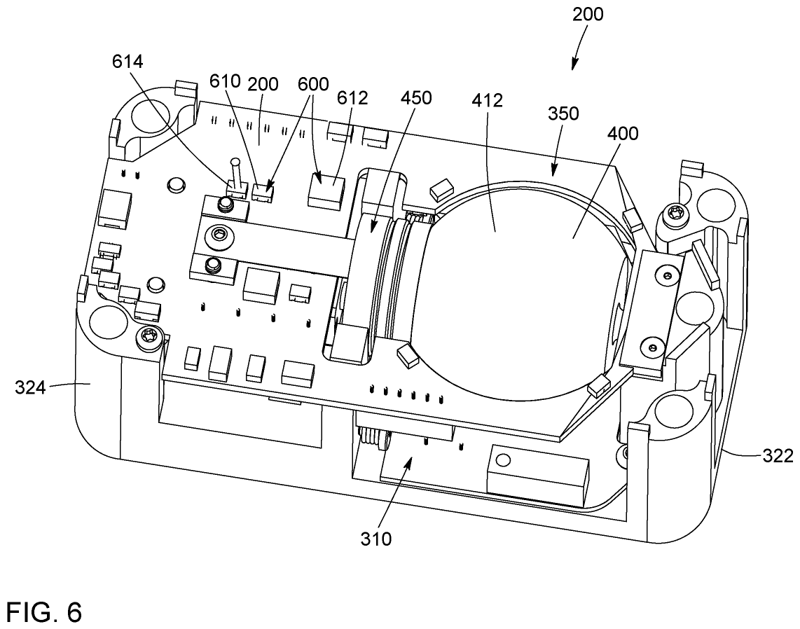

Referring now to the drawings, and more particularly to FIGS. 3 to 7, there is shown an automated optical system 200 (or automated optical detection system 200) for detection of a sanitary condition of a button 400 in accordance with an embodiment. An exemplary elevator button is disclosed in U.S. Pat. No. 9,522,200, the disclosure of which being hereby incorporated by reference in its entirety.

In the embodiment shown, the automated optical detection system 200 comprises a button housing 300 defining a button-receiving cavity 310 and comprising an inner side 312 delimiting at least partially the button-receiving cavity 310. The system 200 further comprises the button 400 disposed at least partially in the button-receiving cavity 310 and being at least partially reachable from an outside of the button housing 300. The button 400 has an outer surface 410 (or user-contacting surface). The system 200 also comprises an optical assembly 500 to capture at least one optical signal of at least one of physical and electrical features of the button 400, a sanitary condition detection assembly 600 to detect a predetermined sanitary condition of the button 400 from information derived from the captured optical signal of the physical and/or electrical features of the button 400; and a disinfecting system 700 for disinfecting the button 400.

In the embodiment shown, the optical assembly 500 may be configured to capture at least one optical signal of the button 400 (for instance but without being limitative an image of a portion 412 of the outer surface 410 of the button 400). The sanitary condition detection assembly 600 may be configured to detect the predetermined sanitary condition on the portion 412 of the outer surface 410 of the button 400 from information derived from the captured optical signal of the button (for instance from the captured image of the portion 412 of the outer surface 410 of the button 400); and the disinfecting system 700 may be for disinfecting at least partially the button 400 (for instance the portion 412 of the outer surface 410 of the button 400).

It is however understood that the present disclosure is not limited to a detection system 200 wherein an image of a portion of the button, for instance of an outer surface thereof, is captured. The detection system might comprise a button sensor assembly, which can be for instance an optical assembly, to detect for instance an electromagnetic field generated by or in the vicinity of the button 400 or any other feature, for instance physical and/or electrical features or the like, related to the sanitary condition of the button, and to capture an optical signal thereof. The term optical should neither be limited to the visible spectrum. The optical assembly may thus be configured to capture for instance and without being limitative holographic and/or interferometric signals of the button corresponding to a whole wavefront of the electromagnetic field having had an interaction with the button. For instance, the disinfecting system 700 is positioned on the inner side 312 of the button housing 300 and is for disinfecting the portion 412 of the outer surface 410 of the button 400 that is exposed on the inner side 312 of the button housing 300.

### Button Housing and Button

In the embodiment shown, the button housing 300 has a substantially parallelepipedal shape and comprises a front panel 320, a rear panel 322 spaced-apart from the front panel 320 and a peripheral wall 324 extending between the front and rear panels 320, 322. The front and rear panels 320, 322 and the peripheral wall 324 delimit together at least partially the button-receiving cavity 310. The front and rear panels 320, 322 and the peripheral wall 324 have each an inner side forming together at least partially the inner side 312 of the button housing 300 and delimiting together at least partially the button-receiving cavity 310 of the button housing 300.

In the embodiment shown, the front panel 320 has an outer surface 323 comprising indicators (for instance floor indicators). For instance, the floor indicators (for instance a portion of the front panel 310) can be made of translucent material and be illuminated from a rear side (or inner side) of the front panel 320 (for instance from the button-receiving cavity) using low intensity lights such as LED in order to inform the user that the floor call has been registered. The floor indicators or information provided on the outer surface 323 may also be in Braille format. For instance, the front panel 320, the rear panel 322 and/or the peripheral wall 324 are removably mounted to each other so as to ease the access to the button-receiving cavity 310, for instance by removing the front panel 320. In the embodiment shown, the automated detection system 200 also comprises a printed circuit board 202 located at least partially in the button-receiving cavity 310, for instance mounted to portions of the peripheral wall 324. Components of the sanitary condition detection assembly 600, the disinfecting system 700 and/or the optical assembly 500 are mounted to the printed circuit board 202. The button 400 is shaped and dimensioned to be at least partially contained—or received—in the button-receiving cavity 310. Moreover, a button-actuating opening 321 is formed in the front panel 320, which is shaped and dimensioned for a front portion of the button 400 to protrude through the front panel 320 towards the user (i.e. to be exposed to the user), so that at least a portion of the button 400 is reachable by the user.

In the embodiment shown, the button-actuating opening 321 is shaped and dimensioned for at least about 10% of a surface area of the outer surface 410 of the button 400 to be exposed. In another embodiment, at least about 20% of the surface area of the outer surface 410 of the button 400 is exposed. In yet another embodiment, at least about 25% of the surface area of the outer surface 410 of the button 400 is exposed.

In the embodiment shown, the button 400 has a rotational symmetry allowing rotation thereof with respect to the button housing 300 about a rotation axis R. In the embodiment shown, the rotation axis R of the button 400 is substantially horizontal. For instance, the button 400 has a substantially spherical shape and the button-actuating opening 321 formed in the front panel 320 is substantially circular.

The system 200 further comprises a rotation mechanism 450 operatively coupled to the button 400 for rotating the button 400 with respect to the button housing 300 about the rotation axis R, at least partially within the button-receiving cavity 310. For instance, the button 400 (or at least the outer surface 410 thereof) is at least partially made of a dielectric material. In another embodiment (not represented), the outer surface of the button is substantially transparent, and the button is substantially hollow.

In the embodiment shown, the detection system 200 further comprises a skirt assembly 350 positioned between the button 400 and the housing 300 for limiting user exposure to the disinfecting system 700. For instance, the skirt assembly 350 protrudes inwardly—with respect to the button-receiving cavity 310—from the inner side of the front panel 320.

It is appreciated that the shape and the configuration of the button housing, as well as the shape, the configuration and the relative arrangement of the button and the different components of the button housing can vary from the embodiment shown.

### Optical Assembly

In the embodiment shown, the optical assembly 500 comprises at least one of a coherent optical system, a holographic optical system, an interferometric optical system and a diffraction optical system. In the embodiment shown, the optical assembly 500 is arranged at least partially in the button-receiving cavity 310 and is not reachable by a user from an outside of the button housing 300. For instance, the optical assembly 500 is arranged on the inner side 312 of the button housing 300.

In the embodiment wherein the button is substantially hollow and transparent, one or more components of the optical assembly and/or one or more components of the sanitary condition detection assembly could be arranged at least partially within the button (i.e. inside the button). It is known that the use of a substantially transparent material would require the use of contrasts in order to capture accurate optical signals of the button (for instance accurate images of the outer surface of the button). In the embodiment shown, the optical assembly 500 is based on at least one of interferometric, holographic and diffraction technologies. Different optical systems could be conceived, which are capable of capturing optical signals (for instance diffraction patterns formed by pathogens on the outer surface of the button 400) in different configurations (dark field, transmission, resonator-mode reflection, and the like).

It is known that pathogens such as viruses and bacteria have a low contrast in terms of intensity, whereas they have a higher contrast in terms of phase. It is also known that in coherent optics, diffraction or interference patterns combine a phase and an intensity of a diffracted field by the pathogens (mutual consistency). An interferometric or holographic-based detection allows to measure independently the intensity and the phase of the fields diffracted by the pathogens. In some embodiments, the phase is quantitatively measured with an accuracy of the order of a few nm or even of the order a few tens of pm. For instance, the optical assembly 500 comprises a light detector and one or a limited number of lenses, the lenses being configured to collect and/or focus a quantity of light sufficient in an area of the light detector, in the form of interference and/or diffraction patterns. In the embodiment shown, the optical assembly 500 uses dark field illumination schemes or a Total Internal Reflection Fluorescence (TIRF) Microscopy scheme. It is known that such schemes render the interference pattern particularly sensible to a presence of pathogens such as microbiological agents on a dielectric surface.

It is appreciated that the shape, the configuration, and the location of the optical assembly 500 with respect to the button housing 300 and the button 400 can vary from the embodiment shown.

### Sanitary Condition Detection Assembly

In the embodiment shown, the sanitary condition detection assembly 600 comprises a processor 610 (or processing unit 610) and a storage medium 612 (or memory 612) having stored thereon processor-readable instructions for processing optical signal of the button (for instance images of the portion 412 of the outer surface 410 of the button 400). The processor-readable instructions are also for comparing the captured optical signal by the optical assembly 500 with optical signatures (or reference optical signals) of cell cultures of viruses and/or bacteria.

The sanitary condition detection assembly 600 can comprise one or more computers or servers, provided with processors, memory and communication interfaces. The sanitary condition detection assembly 600 may reside locally, in the button-receiving cavity 310 of the button housing 300, or at least partially remotely, for instance in a cloud-based implementation. A software application may run on the sanitary condition detection assembly 600. The software application communicates with and controls the optical assembly 500 and/or the rotation mechanism 450. It is also understood that the sanitary condition detection assembly 600 can be shared between a plurality of buttons to be detected and/or controlled. As detailed below, in some embodiments, the optical assembly 500 and/or the sanitary condition detection assembly 600 have access to cloud-based Al-algorithms. In the embodiment shown, the sanitary condition detection assembly 600 comprises a machine-learning algorithm system. For instance, the machine-learning algorithm system comprises one or more convolutional neural networks. In the embodiment shown, the processor-readable instructions are for outputting an indication of whether at least one of a virus and a bacterium is detected onto the outer surface 410 of the button 400 in real-time.

In the embodiment shown, the sanitary condition detection assembly 600 communicates with the optical assembly 500. The optical signals captured by the optical assembly 500 are sent from the optical assembly 500 to the sanitary condition detection assembly 600, via a wired and/or wireless connection, such as Wi-Fi connection, for example. For instance, the sanitary condition detection assembly 600 comprises a communication module 614 including one or more of the following interfaces: a Wi-Fi interface, a Bluetooth interface, a 4G or 5G interface to communicate with the optical assembly 500 and/or to communicate the indication of whether at least one of a virus and a bacterium is detected onto the portion 412 of the outer surface 410 of the button 400. The system may further comprise a button breakage detection system to alert users of a breakage and/or a dysfunction of the detection system, for instance via the communication module.

The sanitary condition detection assembly 600 can access, via its communication module, remote machine training algorithms that have been previously trained to detect viruses and/or bacteria. Alternately, it could be conceived an automated optical detection system wherein the trained Al-algorithms would be stored and executed by the sanitary condition detection assembly 600. The sanitary condition detection assembly 600 may have access to reference optical signatures (for instance image data) from bacteria and/or viruses on a similar material (for instant a dielectric material) as the one in which the outer surface 410 of the button 400 is at least partially formed. In the embodiment shown, real-time optical data of the portion 412 of the outer surface 410 of the button 400 by the optical assembly 500 are communicated to the sanitary condition detection assembly 600 which may store and analyze the optical data to train the components of the machine-learning algorithm system.

Once the machine-learning algorithm system will be trained, the automated optical detection system 200 will be able to continuously detect and identify pathogens on the outer surface 410 of the button 400, provide inspection reports in real-time and, if need be, generate alerts as a function of pre-determined criteria, for instance to take cleaning measures and/or preventive and/or care administering measures, in order to prevent or limit the risk of disease outbreaks. The automated optical detection system 200, for instance the sanitary condition detection assembly 600 thereof via its communication module, is configured to communicate with a database comprising pathogen data. For instance, the database will be populated via sampling and analysing pathogens which can be detected in health centers and/or hospitals. In other words, the detection system will allow the population and providing of a new catalog of pathogen signatures (i.e. to obtain specific patterns for identified bacteria and/or viruses). For instance, identified virus and/or bacterium colonies are grown in laboratories on similar buttons and/or on a material substantially similar to the material in which the button 400 is at least partially formed. The virus and/or bacterium colonies are then studied and/or analyzed continuously and/or at different time intervals for instance with similar holographic and/or interferometric and/or diffraction optical systems as the ones of the optical assembly 500 and with other optical systems, such as, for instance, multimodal high-definition microscopy approaches combining for instance spectroscopic digital holographic microscopy approaches with fluorescent imaging (such as immunohistochemistry). With multimodal high-definition microscopy approaches, reference optical signatures of the observed colonies can be obtained.

By applying different approaches on the same colonies, a highly accurate and specific optical signature (the reference optical signature) corresponding to the observed diffraction pattern for each colony can be obtained. Accurate data with respect to the composition of the observed pathogens onto the outer surface of the button 400 can be obtained via immunohistochemistry or any other suitable processes. The above-mentioned database may be used for the training of the machine training algorithms.

Different automated learning approaches—for instance deep learning approached—may be used. For instance, the machine-learning algorithm system comprises one or more convolutional neural networks. Image-to-image translation approaches may be used, to train a Generative Adversarial Network (GAN) to predict high-resolution images on the basis of diffraction patterns. GAN are described for in instance in Goodfellow, Ian, et al. “Generative adversarial nets”, Advances in neural information processing systems, 2014. To this end, an architecture based for instance on the one disclosed in Isola, Phillip, et al. “Image-to-image translation with conditional adversarial networks”, IEEE Conference on Computer Vision and Pattern Recognition, 2017 can be adapted to the specificities of the data (such as resolution, number of convolutional neural layers and the like).

In the embodiment shown, a discriminative neural network may also be used to predict directly a type of virus and/or bacterium colony in the considered sample. The discriminative neural network may be trained via a given reference optical signature or via a diffraction pattern. An architecture similar to the one disclosed in He, Kaiming, et al. “Deep residual learning for image recognition”, Proceedings of the IEEE conference on computer vision and pattern recognition, 2016, or in Yu, Fisher, Vladlen Koltun, and Thomas Funkhouser “Dilated residual networks”, Proceedings of the IEEE conference on computer vision and pattern recognition, 2017 may be used. In other words, the sanitary condition detection assembly 600 is configured to detect different lighting signatures on the outer surface 410 of the button 400. For instance, the sanitary condition detection assembly 600 comprises a complementary metal-oxide-semiconductor (CMOS) sensor with a high resolution and a broad dynamic range and enables recording optical signals produced by the optical assembly 500. The sanitary condition detection assembly 600 is also configured to distinguish in the interference fringes signatures corresponding to one or more pathogens, such as viruses and/or bacteria. The CMOS sensor may comprise up to several million pixels and is configured to transform a light received on its pixels into a plurality of bits.

The sanitary condition detection assembly 600 may further comprise a programmable gate array such as, for instance, a Field-programmable gate array FPGA configured to treat a continuous data flow produced by the pixels of the CMOS sensor. The sanitary condition detection assembly 600 may further comprise one or more complementary digital modules, such as a microcontroller and an embedded RAM memory, for instance to send—via Wi-Fi connection or the like—the data flow toward a cloud computing device for analyzing, monitoring and storing the data. Using a Field-programmable gate array may provide an edge-computing environment and may allow to extract selective features from the interference patterns in order to reduce a quantity of the data sent toward the cloud computing device.

It is appreciated that the shape, the configuration, and the technologies used by the sanitary condition detection assembly 600 can vary from the embodiment shown.

### Disinfecting System

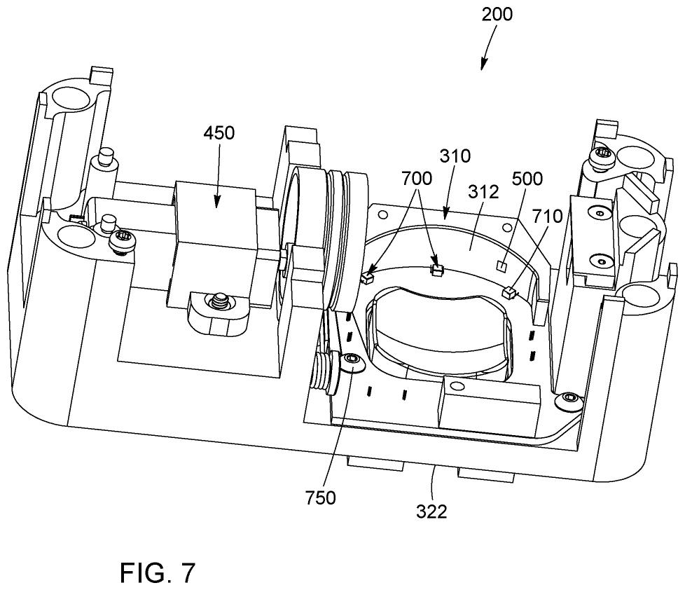

In the embodiment shown, the disinfecting system 700 is positioned 13 or arranged—on the inner side 312 of the button housing 300 and is for disinfecting the portion 412 of the outer surface 410 of the button 400 that is exposed on a corresponding portion of the inner side 312 of the button housing 30. In the embodiment shown, the disinfecting system 700 comprises one or more ultra-violet germicidal lamps 710 (for instance one or more UVC LEDs). In the embodiment shown, as represented in FIG. 7, the disinfecting system 700 comprises three germicidal UV lamps 710 located at a junction of the inner sides of the rear panel 322 and the peripheral wall 324, in an upper portion of the inner side 312 of the button housing 300. In other words, in the embodiment shown, the disinfecting system 700 is arranged substantially above a rear portion of the outer surface 410 of the button 400, when the button 400 is rotatably mounted to the button housing 300 about a substantially horizontal rotation axis R.

The disinfecting system 700 of the present disclosure aims at sanitizing the buttons by shining intense shortwave UV light to the exposed button outer surface 410. Ultraviolet light kills microorganisms by damaging their DNA. UV photons of wavelength comprised between about 200 nm and about 280 nm—for instance a wavelength around 260 nm—have enough energy to disrupt the chemical bonds that hold the building blocks of DNA together. Specifically this short-wave ultraviolet light disrupts DNA base pairing causing thymine-thymine dimers. If the damage is severe enough, the exposed microorganism cannot repair the damage and rapidly dies. Ultraviolet light affects living organisms but otherwise leaves inorganic material intact. Nothing is emitted except electromagnetic energy. UV radiation is thus preferable over chemical means of sterilization when chemical residues can accumulate and cannot be removed efficiently. In the embodiment shown, the ultra-violet germicidal lamp 710 of the disinfecting system 700 generates a disinfecting light having a spectral profile ranging from about 200 nm to about 280 nm. In another embodiment, the spectral profile of the disinfecting light ranges from about 220 nm to about 270 nm. In yet another embodiment, the spectral profile of the disinfecting light ranges from about 250 nm to about 260 nm. In the embodiment shown, the disinfecting system 700 is configured to eliminate more than about 90% of pathogens (for instance bacteria and/or viruses) onto the outer surface 410 of the button 400 exposed to the light of the disinfecting system 700. In another embodiment, the disinfecting system 700 is configured to eliminate more than about 99% of pathogens onto the outer surface 410 of the button 400.

In the embodiment wherein the detection system 200 comprises a skirt assembly 350 positioned between the button 400 and the housing 300, the skirt assembly 350 is shaped and positioned so as to prevent exposure of human operators to the UV light. Exposure to UV light can cause eye and skin damage in humans. Thus it is most important to find ways to shield the UV emission using opaque baffles and obstacles. Organizations such as US National Institute for Occupational Safety and Health (NIOSH) recommends that the time of exposure to an intensity of 100 microwatts per square centimeter at wavelength 254 nanometers not exceed 1 minute. For instance, a duration of an exposure of the outer surface 410 of the button 400 to the light of the disinfecting system 700 is shorter than about 180 seconds. In another embodiment, the duration of the exposure of the outer surface 410 of the button 400 to the light of the disinfecting system 700 is about 120 seconds.

It is appreciated that the shape, the configuration, and the location of the disinfecting system 700, as well as the number, the shape and the location of the ultra-violet germicidal lamp 710 thereof can vary from the embodiment shown. Moreover, the germicidal UV (Ultraviolet) lights may be used for sanitizing buttons with or without the use of additional disinfection method such chemical disinfectants.

### Additional Features of the System

As mentioned above, in the embodiment shown, the button 400 is an elevator button and the button housing 300 is an elevator button panel. The automated optical detection system 200 may further comprise a sensor 750 positioned proximate the button for detecting actuation thereof. In the embodiment shown, the sensor 750 is operatively coupled with at least one of the optical assembly 500 and the disinfecting system 700. The sensor 750 may also be operatively coupled with the rotation mechanism 450. It could also be conceived a detection system wherein the sanitary condition detection assembly 600 could be actuated remotely, for instance to proceed to the detection of a sanitary condition on the button and/or the disinfecting of the outer surface of the button even though the button is not actuated.

The purpose of the rotation mechanism is to slowly rotate the button 400 so as to allow optical signals of surfaces touched by users to be captured by the optical assembly 500, to be detected/tested/controlled by the sanitary condition detection assembly 600 and to be sanitized by the disinfecting system 700. The rotation of the button 300 can be performed in a few different ways. One method could consist in imparting a slow, nearly imperceptible, substantially constant rotation so as to make the button 300 appear stationary to the users. This approach has the advantage of avoiding fingers being caught on the entering edge of the button. The rotation speed can be selected in the range between one rotation about every 5 minutes to two rotations per minute so as to provide a safe operation and a rapid constant sanitizing of the surface contaminated by the users. Considering that, once pressed, a floor button—in the embodiment wherein the button is an elevator button—would normally not be pressed again until the called floor has been reached, the probability of one person touching a surface that was already pressed is low.

### General Principle

It is thus understood, as described above, that the automated optical detection system 200 in accordance with the present disclosure is configured to provide a smart button assembly—for instance a smart elevator button assembly, in the embodiment shown—which is able to detect, in a localized and prompt manner, epidemy outbreak risks and by doing so to limit and/or stop a spreading of infection chains. In other words, the automated optical detection system 200 is configured to measure and communicate in real-time parameters corresponding to pathogens detected on the button.

The detection system is configured so that an identification of the microbes based on machine learning models will be substantially instantaneous to yield an answer in an order of a fraction of a second. In other words, individuals carrying virulent or dangerous microbes could be intercepted “on the fly” while they are still in the elevator or in the vicinity of the button. This is particularly appropriate in health institutions such as hospitals when trained employees and medical facilities are available for quarantining, further analysis, and treatment. This capability allows reducing and/or stopping transmission of diseases by quarantining source individuals immediately. The automated optical detection system 200 is shaped and dimensioned to be contained in a limited space, allows accurate and specific measurements without requiring the use of markers and is configured to communicate the measurements in real-time.

It is understood that elevator buttons are particularly relevant statistic measure tools since they form a required step of a use of an elevator: users usually have to press an elevator button to enter an elevator and/or to go to a particular floor.

### Automated Optical Detection Method

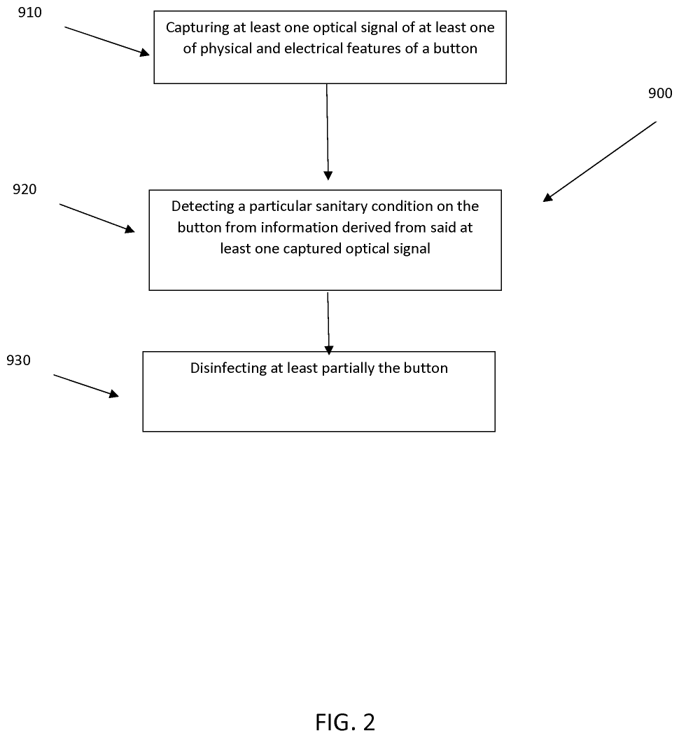

According to another aspect of the disclosure, as represented in FIG. 2, there is provided an automated optical method 900 for detecting a sanitary condition on a button, for instance on an outer surface thereof. The method according to embodiments of the present disclosure may be carried out with an automated optical detection system 200 as the one described above with reference to FIGS. 3 to 7.

In the embodiment shown, the method 900 comprises a step 910 of capturing at least one optical signal of at least one of physical and electrical features of the button 400 (for instance an image of a portion 412 of an outer surface 410 thereof); a step 920 of detecting a particular sanitary condition of the button 400 (for instance on the portion of the outer surface thereof) from (or a step 920 of determining whether the button has a particular sanitary condition based on) information derived from the captured optical signal of the physical and/or electrical feature of the button; and a step 930 of disinfecting at least partially the button 400 (for instance the portion of the outer surface thereof). In the embodiment shown, the method 900 further comprises providing for output an indication of whether at least one of a virus and a bacterium is detected onto the portion of the outer surface of the button 400 in real-time. In the embodiment shown, the indication of whether the at least one of a virus and a bacterium is detected is communicated using one or more of the following interfaces: a Wi-Fi interface, a Bluetooth interface, a 4G or 5G interface. Different users (such as relevant services of a hospital) could receive automated notifications, for instance via Web-platforms, text messages and/or dedicated applications and/or software.

### Step of Detecting the Particular Sanitary Condition

In the embodiment shown, the step 920 of detecting the particular sanitary condition (or step of determining whether the button has the particular sanitary condition) is achieved by comparing the captured optical signals with optical signatures (or reference optical signals) of cell cultures of at least one of viruses and bacteria. In the embodiment wherein the button 400 is at least partially made of a dielectric material, the cell cultures which are captured to provide the reference optical signals are carried out on a dielectric material similar to the dielectric material of the button 400.

In the embodiment shown, the reference optical signatures of cells cultures are carried out by at least one of holographic optical system, an interferometric optical system, a digital holographic microscopy and a diffraction optical system. For instance, the step 920 of detecting the particular sanitary condition comprises using a machine- learning algorithm system comprising, for instance, one or more convolutional neural networks. In the embodiment shown, the step 920 of detecting the particular sanitary condition further comprises training the machine-learning algorithm system to identify one or more viruses and/or bacteria.

### Step of Capturing the Optical Signal of the Button

In the embodiment shown, the step 910 of capturing optical signals of the button 400 comprises using at least one of a holographic optical system, an interferometric optical system and a diffraction optical system.

### Step of Disinfecting the Button

In the embodiment shown, the step 930 of disinfecting at least partially the portion of the outer surface of the button 400 is carried out after the step 910 of capturing the optical signals of the button. By doing so, a detection of the pathogens on the outer surface 410 of the button 400 is not compromised by the disinfecting thereof.

It could also be conceived a method wherein optical signals of the outer surface of the button would be captured while the outer surface is disinfected, in order to capture optical signals of pathogens in different states (for instance while the pathogens are alive, being killed, killed and the like), and thus more easily distinguish patterns from picture noise. An accelerated observation of the different states of the pathogens may form an evolution pattern for specific pathogens (i.e. transformational patterns). A plurality of optical devices may thus be used and placed in specific locations of the system to allow capturing optical signals of the pathogens in different states. The present invention will also allow distinguishing noise generated by dust, grease, organic material on the user finger and the like and/or acceptable bacteria from dangerous ones.

In the embodiment shown, the step 930 of disinfecting the button 400 is carried out via ultra-violet. It could also be conceived a method wherein, when a unidentified virus and/or bacterium is detected onto the button (i.e. when a pathogen which does not correspond to previously identified pathogens is detected), the step of disinfecting the button is postponed in order to maintain the corresponding portion of the button unexposed (i.e. within the button-receiving cavity) until an alerted user (for instance alerted via a text message, an email or any other communication sent by the communication module) comes and collects the corresponding sample for further analysis thereof. It could also be conceived a method wherein, when a non-identified virus and/or bacterium is detected onto the button, additional optical signals are captured by the optical assembly (for instance optical signals of other portions of the button). For instance, information corresponding to this non-identified pathogen could be stored and used to improve the sanitary condition detection assembly (for instance to further train the machine-learning algorithm thereof). In other words, the method could be configured to be self-improved.

### Step of Rotating the Button in the Button-Receiving Cavity

In the embodiment shown, the button is mounted to a button housing 300 defining a button-receiving cavity 310, and the method 900 also comprises a step of rotating the button 400 in the button-receiving cavity. For instance, the method 900 comprises configuring the button 400 in a first angular configuration in the button-receiving cavity 310 wherein the portion of the outer surface 410 of the button 400 is at least partially exposed for a user to actuate the button 400 (i.e. to exert a pressure thereon); and further comprises configuring the button 400 in a second angular configuration in the button-receiving cavity 310 wherein the optical signals of the button (for instance the images of the portion of the outer surface of the button) are captured.

In the embodiment shown, the method 900 further comprises configuring the button 300 in a third angular configuration wherein the portion of the outer surface is disinfected.

### Additional Steps of the Method

In the embodiment wherein the button 400 is an elevator button, the method 900 may further comprise detecting an actuation of the button 400. It is appreciated that the number and the order of the steps of the method can vary from the embodiment shown. Meta-analyses could also be carried out, in order to compare different elevators, hospitals, buildings, cities, countries and the like and/or to compare outbreaks of bacteria and/or viruses as a function of time, use frequency and the like. Such meta-analyses could be used to quantify an efficiency of the system and an efficiency of sanitary measures taken by the different places equipped with the system. Moreover, traceability of the detected pathogens could further be improved with an operatively coupling of the sanitary condition detection assembly with facial recognition surveillance camera systems and/or with access card and/or smart phone identification systems and the like. In the embodiment wherein individuals in a building are identified and located via access cards or other biometric methods, the detection system can be used to associate the detected pathogen with individuals. For example, if a user such as a health care worker uses a RFID card to call an elevator or unlock the access to specific levels of a building, any pressing of elevator buttons leading to the identification of a specific pathogen could result either in messaging the individual to warn of a potential infection or a request to go seek medical help. The authorities responsible for health or security as well as the supervisor of a potentially infected individual may also receive messages to inform them of the situation, so they can take the appropriate actions. Alternatively, the elevator panels could also contain a video recording device that substantially continuously saves the video footage in a buffer. If a pathogen with potential health consequences is detected, the content of the video buffer could be analyzed to automatically identify the individual that activated the button or the video segment could be sent to the authorities responsible for health and security for the adequate corrective action to be taken. Forecast models could be determined on the basis of the collected information to forecast disease and/or infection outbreak.

Several alternative embodiments and examples have been described and illustrated herein. The embodiments of the invention described above are intended to be exemplary only. A person of ordinary skill in the art would appreciate the features of the individual embodiments, and the possible combinations and variations of the components. A person of ordinary skill in the art would further appreciate that any of the embodiments could be provided in any combination with the other embodiments disclosed herein. It is understood that the invention may be embodied in other specific forms without departing from the central characteristics thereof. The present examples and embodiments, therefore, are to be considered in all respects as illustrative and not restrictive, and the invention is not to be limited to the details given herein. Accordingly, while the specific embodiments have been illustrated and described, numerous modifications come to mind. The scope of the invention is therefore intended to be limited by the scope of the appended claims.

1. An automated optical method for detecting a sanitary condition on a button, the method comprising:
capturing at least one optical signal of at least one of physical and electrical features of the button;
determining whether the button has a particular sanitary condition based on information derived from said at least one captured optical signal of said at least one of physical and electrical features of the button; and
disinfecting at least partially the button.

2. The method according to claim 1, further comprising providing for output an indication of whether at least one of a virus and a bacterium is detected onto the button in real-time.

3. The method according to claim 2, wherein the indication of whether said at least one of a virus and a bacterium is detected is communicated using one or more of the following interfaces: a Wi-Fi interface, a Bluetooth interface, a 4G interface or a 5G interface.

4. The method according to any one of claims 1 to 3, wherein determining whether the button has the particular sanitary condition is achieved by comparing said at least one captured optical signal with optical signatures of cell cultures of at least one of viruses and bacteria.

5. The method according to claim 4, wherein the button is at least partially made of a dielectric material and wherein said cell cultures are carried out on a dielectric material similar to the dielectric material of the button.

6. The method according to claim 4 or 5, wherein said optical signatures of cell cultures are carried out by at least one of holographic optical system, an interferometric optical system, a digital holographic microscopy and a diffraction optical system.

7. The method according to any one of claims 4 to 6, wherein detecting the particular sanitary condition comprises using a machine-learning algorithm system.

8. The method according to claim 7, wherein the machine-learning algorithm system comprises one or more convolutional neural networks.

9. The method according to claim 7 or 8, further comprising training the machine-learning algorithm system to identify said at least one of a virus and a bacterium.

10. The method according to any one of claims 1 to 9, wherein capturing said at least one optical signal of the button comprises using a coherent optical system.

11. The method according to any one of claims 1 to 10, wherein capturing said at least one optical signal of the button comprises using at least one of a holographic optical system, an interferometric optical system and a diffraction optical system.

12. The method according to any one of claims 1 to 11, wherein the button is rotatably mounted to a button housing defining a button-receiving cavity and wherein the method further comprises:
configuring the button in a first angular configuration in the button-receiving cavity wherein the button is at least partially exposed; and
configuring the button in a second angular configuration in the button-receiving cavity wherein the at least one optical signal of the button is captured.

13. The method according to claim 12, further comprising configuring the button in a third angular configuration wherein the button is disinfected.

14. The method according to claim 13, wherein the disinfecting is carried out after the capturing of said at least one optical signal.

15. The method according to any one of claims 1 to 14, wherein the disinfecting is carried out via ultra-violet.

16. The method according to any one of claims 1 to 15, wherein the button is an elevator button.

17. The method according to any one of claims 1 to 16, further comprising detecting an actuation of the button.

18. An automated optical detection system for detection of a sanitary condition of a button, the system comprising:
a button housing defining a button-receiving cavity and comprising an inner side;
at least one button disposed at least partially in the button-receiving cavity;
an optical assembly to capture at least one optical signal of at least one of physical and electrical features of the button;
a sanitary condition detection assembly to detect a predetermined sanitary condition of the button from information derived from said at least one captured optical signal of said at least one of physical and electrical features of the button; and
a disinfecting system for disinfecting the button.

19. The system according to claim 18, wherein the sanitary condition detection assembly comprises a processor and a storage medium having stored thereon processor-readable instructions for processing the at least one captured optical signal of the button and comparing said at least one captured optical signal with optical signatures of cell cultures of at least one of viruses and bacteria.

20. The system according to claim 19, wherein the sanitary condition detection assembly comprises a machine-learning algorithm system.

21. The system according to claim 20, wherein the machine-learning algorithm system comprises one or more convolutional neural networks.

22. The system according to any one of claims 19 to 21, wherein the processor-readable instructions are for outputting an indication of whether at least one of a virus and a bacterium is detected on the button in real-time.

23. The system according to claim 22, wherein the sanitary condition detection assembly comprises a communication module including one or more of the following interfaces: a Wi-Fi interface, a Bluetooth interface, a 4G interface or a 5G interface to communicate the indication of whether said at least one of a virus and a bacterium is detected on the button.

24. The system according to any one of claims 18 to 23, wherein the optical assembly comprises a coherent optical system.

25. The system according to any one of claims 18 to 24, wherein the optical assembly comprises at least one of a holographic optical system, an interferometric optical system and a diffraction optical system.

26. The system according to any one of claims 18 to 25, wherein the at least one button has a rotational symmetry allowing rotation thereof with respect to the button housing about a rotation axis, the system further comprising a rotation mechanism operatively coupled to the button for rotating the button with respect to the button housing.

27. The system according to claim 26, wherein the rotation axis is substantially horizontal.

28. The system according to any one of claims 18 to 27, wherein the button has an outer surface, the optical assembly being for capturing at least one image of a portion of the outer surface of the button.

29. The system according to claim 28, wherein the outer surface of the button is substantially transparent.

30. The system according to any one of claims 18 to 29, wherein the button is substantially hollow.

31. The system according to claim 30, wherein the optical assembly is arranged at least partially within the button.

32. The system according to any one of claims 18 to 31, wherein the disinfecting system is positioned on the inner side of the button housing.

33. The system according to claim 32, wherein the disinfecting system comprises an ultra-violet germicidal lamp.

34. The system according to claim 33, wherein the ultra-violet germicidal lamp generates a disinfecting light having a spectral profile ranging from about 200 nm to about 280 nm.

35. The system according to any one of claims 18 to 34, wherein the button is an elevator button and the button housing is an elevator button panel.

36. The system according to claim 35, further comprising a sensor positioned proximate said at least one button for detecting actuation thereof.

37. The system according to claim 36, wherein the sensor is operatively coupled with at least one of the optical assembly and the disinfecting system.
